(19) United States
(12) Patent Application Publication
(10) Pub. No.: US 2010/0152831 A1
(43) Pub. Date: Jun. 17, 2010

(54) Implantable Medical Devices Having Multiblock Copolymers

(75) Inventors: Ya Guo, Cotati, CA (US); Kishore Udipi, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/333,642

(22) Filed: Dec. 12, 2008

(51) Int. Cl. A61F 2/82; A61F 2/00; A61F 2/06; A61F 2/92; A61B 17/04; A61F 2/24; A61M 1/00; A61N 1/36; A61B 17/58; A61P 43/00

(52) U.S. Cl. 623/1.13; 424/423; 623/1.42; 623/1.2; 623/1.49; 606/231; 623/2.42; 604/8; 607/9; 606/331

(57) ABSTRACT

Provided herein are implantable medical devices comprising a biodegradable multiblock copolymer comprising at least three blocks; wherein the at least three blocks includes at least one inner block and two end blocks; further wherein each of the at least one inner block comprises monomers selected from the group consisting of e-caprolactone, r-butylactone, trimethylene carbonate, caprolactone derivatives, P-Dioxanone, and combinations thereof; and further wherein each of the end blocks comprises monomers selected from the group consisting of l-lactide, D-lactide, glycolide, L,D-lactide, and combinations thereof.

### FIELD OF THE INVENTION

The present disclosure generally relates to implantable medical devices having biodegradable multiblock copolymers useful for treating various diseases and conditions.

### BACKGROUND OF THE INVENTION

Cardiovascular disease, specifically atherosclerosis, remains a leading cause of death in developed countries. Atherosclerosis is a multifactorial disease that results in a narrowing, or stenosis, of a vessel lumen. Briefly, pathologic inflammatory responses resulting from vascular endothelium injury causes monocytes and vascular smooth muscle cells (VSMCs) to migrate from the sub endothelium and into the arterial wall's intimal layer. There the VSMC proliferate and lay down an extracellular matrix causing vascular wall thickening and reduced vessel patency.

Cardiovascular disease caused by stenotic coronary arteries is commonly treated using either coronary artery by-pass graft (CABG) surgery or angioplasty. Angioplasty is a percutaneous procedure wherein a balloon catheter is inserted into the coronary artery and advanced until the vascular stenosis is reached. The balloon is then inflated restoring arterial patency. One angioplasty variation includes arterial stent deployment. Briefly, after arterial patency has been restored, the balloon is deflated and a vascular stent is inserted into the vessel lumen at the stenosis site. The catheter is then removed from the coronary artery and the deployed stent remains implanted to prevent the newly opened artery from constricting spontaneously. However, balloon catheterization and stent deployment can result in vascular injury ultimately leading to VSMC proliferation and neointimal formation within the previously opened artery. This biological process whereby a previously opened artery becomes re-occluded is referred to as restenosis.

The introduction of intracoronary stents into clinical practice has dramatically changed treatment of obstructive coronary artery disease. Since having been shown to significantly reduce restenosis as compared to percutaneous transluminal coronary angioplasty (PTCA) in selected lesions, the indication for stent implantation was been widened substantially. As a result of a dramatic increase in implantation numbers worldwide in less selected and more complex lesions, in-stent restenosis (ISR) has been identified as a new medical problem with significant clinical and socioeconomic implications. ISR is due to a vascular response to injury, and this response begins with endothelial denudation and culminates in vascular remodeling after a significant phase of smooth muscle cell proliferation.

Additionally, recent advances in in situ drug delivery have led to the development of implantable medical devices specifically designed to provide therapeutic compositions to remote anatomical locations. Perhaps one of the most exciting areas of in situ drug delivery is in the field of intervention cardiology. Vascular occlusions leading to ischemic heart disease are frequently treated using percutaneous transluminal coronary angioplasty (PTCA) whereby a dilation catheter is inserted through a femoral artery incision and directed to the site of the vascular occlusion. The catheter is dilated and the expanding catheter tip (the balloon) opens the occluded artery restoring vascular patency. Generally, a vascular stent is deployed at the treatment site to minimize vascular recoil and restenosis. However, in some cases stent deployment leads to damage to the intimal lining of the artery which may result in vascular smooth muscle cell hyperproliferation and restenosis. When restenosis occurs it is necessary to either re-dilate the artery at the treatment site, or, if that is not possible, a surgical coronary artery bypass procedure must be performed.

Generally, implantable medical devices are intended to serve long term therapeutic applications and are not removed once implanted. In some cases it may be desirable to use implantable medical devices for short term therapies. However, their removal may require highly invasive surgical procedures that place the patient at risk for life threatening complications. Therefore, it would be desirable to have medical devices designed for short term applications that degrade via normal metabolic pathways and are reabsorbed into the surrounding tissues.

In general, polymer selection criteria for use as biomaterials are to match the mechanical properties of the polymer(s) and degradation time to the needs of the specific in vivo application. The factors affecting the mechanical performance of biodegradable polymers are those that are well known to the polymer scientist, and include monomer selection, initiator selection, polymerization conditions, process conditions and the presence of additives. These factors in turn influence the polymer's hydrophilicity, crystallinity, melt and glass-transition temperatures, molecular weight, molecular-weight distribution, end groups, sequence distribution (random versus blocky) and presence of residual monomer or additives. In addition, the polymer scientist working with biodegradable materials must evaluate each of these variables for its effect on biodegradation. Currently known biodegradable polymer's physical characteristics are difficult to modify, or tune, to match specific clinical demands.

Implanted medical devices that are made from or coated with biodegradable biocompatible polymers offer substantial benefits to the patient. Reduced inflammation and immunological responses promote faster post-implantation healing times in contrast to uncoated medical devices. Polymer-coated vascular stents, for example, may encourage endothelial cell proliferation and therefore integration of the stent into the vessel wall. Loading the coating polymers with appropriate drugs is also advantageous in preventing undesired biological responses. For example, an implanted polylactic acid polymer loaded with hirudin and prostacyclin does not generate thrombosis, a major cause of post-surgical complications (Eckhard et al, Circulation, 2000, pp 1453-1458).

Thus, there is a need for improved polymeric materials which are suitable for forming or coating implantable medical devices. The implantable polymeric materials should be capable of delivering hydrophilic and hydrophobic drugs, effectively coat or fabricate medical devices and be biodegradable.

### SUMMARY OF THE INVENTION

The present disclosure addresses these and other objectives by providing polymers that are biocompatible, biodegradable, and suitable for forming and coating implantable medical devices.

Accordingly, provided herein are implantable medical devices comprising a biodegradable multiblock copolymer comprising at least three blocks; wherein the at least three blocks includes at least one inner block and two end blocks; further wherein each of the at least one inner block comprises monomers selected from the group consisting of e-caprolactone, r-butylactone, trimethylene carbonate, caprolactone derivatives, P-Dioxanone, and combinations thereof; and further wherein each of the end blocks comprises monomers selected from the group consisting of l-lactide, D-lactide, glycolide, L,D-lactide, and combinations thereof.

In one embodiment of the presently disclosed implantable medical device, one of the at least one inner block is a center block.

In another embodiment of the presently disclosed implantable medical device, each block is a copolymer.

In another embodiment of the presently disclosed implantable medical device, each block is a homopolymer.

In another embodiment of the presently disclosed implantable medical device, the center block is a soft block.

In another embodiment of the presently disclosed implantable medical device, each of the end blocks is a hard block.

In another embodiment of the presently disclosed implantable medical device, the blocks alternate between a hard block and a soft block; and the center block is a soft block and each of the end blocks is a hard block.

In another embodiment of the presently disclosed implantable medical device, the multiblock copolymer is a triblock copolymer.

In another embodiment of the presently disclosed implantable medical device, the center block is poly(trimethylenecarbonate) and the end blocks are poly(L-lactide-co-glycolide).

In another embodiment of the presently disclosed implantable medical device, the biodegradable multiblock copolymer is cross-linked.

In another embodiment of the presently disclosed implantable medical device, the end blocks serve as one or more cross-linking points.

In another embodiment of the presently disclosed implantable medical device, the multiblock copolymer is an elastomer.

In another embodiment of the presently disclosed implantable medical device, the implantable medical device further comprises at least one bioactive agent.

In another embodiment of the presently disclosed implantable medical device, the at least one bioactive agent is selected from the group consisting of an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, and combinations thereof.

In another embodiment of the presently disclosed implantable medical device, the bioactive agent is zotarolimus.

In another embodiment of the presently disclosed implantable medical device, the implantable medical device is selected from the group consisting of a vascular stent, stent graft, urethral stent, biliary stent, suture, ocular device, heart valve, shunt, pacemaker, bone screw, bone anchor, protective plate, prosthetic device, and combinations thereof.

In another embodiment of the presently disclosed implantable medical device, the implantable medical device is a vascular stent.

In another embodiment of the presently disclosed implantable medical device, the implantable medical device is self-expanding.

The present disclosure also relates to a self-expanding vascular stent consisting essentially of a biodegradable triblock copolymer which consists essentially of a poly(trimethylene carbonate) center block and two poly(L-lactide-co-glycolide) end blocks. In one embodiment, the self-expanding vascular stent further consists essentially of a bioactive agent.

### DETAILED DESCRIPTION OF THE INVENTION

The present disclosure generally relates to implantable medical devices comprising a biodegradable multiblock copolymer comprising at least three blocks; wherein the at least three blocks includes at least one inner block and two end blocks; further wherein each of the at least one inner block comprises monomers selected from the group consisting of e-caprolactone, r-butylactone, trimethylene carbonate, caprolactone derivatives, P-Dioxanone, and combinations thereof; and further wherein each of the end blocks comprises monomers selected from the group consisting of I-lactide, D-lactide, glycolide, L,D-lactide, and combinations thereof.

The implantable medical devices as claimed herein contain the present multiblock copolymers which are biodegrable. The instant multiblock copolymers may be part of the implantable medical devices or be the material from which implantable devices are fabricated. Thus, an implantable device may comprise, consist of, or consist essentially of the present multiblock copolymers.

Because the presently disclosed multiblock polymers are biodegradable, implantable medical devices which include them may partially or completely biodegrade inside the body of a mammal, for example, a human. As used herein, “biodegradable” refers to a polymeric composition that is biocompatible and subject to being broken down in vivo through the action of normal biochemical pathways. Biodegradable polymers may or may not be reabsorbed into surrounding tissues, however all bioresorbable polymers are considered biodegradable. The biodegradable polymers of the present disclosure are capable of being cleaved into biocompatible byproducts through chemical- or enzyme-catalyzed hydrolysis. The definitions provided in the present disclosure are done so for the avoidance of doubt. Words or terms not specifically defined shall have the ordinary meaning as known to those skilled in the art of implantable medical devices and polymer chemistry.

Further, as used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

In accordance with the scope and teachings of the present disclosure, the biodegradable multiblock copolymers have at least three blocks. The presently disclosed multiblock polymers may have blocks that are each a copolymer or a homopolymer. Homopolymers are polymers resulting from the polymerization of a single monomer and this is a polymer consisting substantially of a single type of repeating unit. A copolymer is a polymer derived from two (or more) monomeric species.

When a multiblock polymer in accordance with the present disclosure is represented by ABBBA, A serves as both end blocks. B is a center block and B also represents the other inner blocks. A or B itself can be a copolymer or a homopolymer. When it is a copolymer it may be composed of two or more different monomeric species but when it is a homopolymer it is composed of one monomeric specie.

Even when each block is a homopolymer, the at least one block will have a different overall monomer constitution because one block will be have a different constitution as compared to at least one other block. The different constitution allows the multiblock polymer to be a multiblock copolymer. It is also within the scope and teachings of the present disclosure that each block itself is a copolymer. Further, the instant multiblock copolymers of the present disclosure may have be entirely made of blocks which are homopolymers or copolymers. Also, the multiblock copolymers may partially be made of homopolymer blocks and partially be made of copolymer blocks.

A polymer is composed of monomers. Therefore, the present block polymers which make up biodegradable multiblock polymers are also ultimately made of monomers. In accordance with the scope and teachings of the present disclosure, in one embodiment, any of the block polymers may be composed of monomers selected from the group consisting of e-caprolactone, r-butylactone, trimethylene carbonate, caprolactone derivatives, P-Dioxanone, l-lactide, D-lactide, glycolide, and L,D-lactide.

The multiblock copolymers disclosed herein have at least three blocks, two of which are end blocks. End blocks are the terminating blocks for the instant multiblock copolymers. As an example, for a multiblock copolymer represented by BABCDEB each letter represents a block. B thus serves as the end blocks. Further, as an example, for a multiblock polymer represented by ABCDEFG, A and G serve as the end blocks.

Within the scope and teachings of the present disclosure an inner block is a block which is not an end block for a multiblock copolymer. Thus, in the above examples, the inner blocks are ABCDE and BCDEF, respectively.

In another embodiment, each of the inner blocks is a polymer which is composed of monomers that may be e-caprolactone, r-butylactone, trimethylene carbonate, caprolactone derivatives, P-Dioxanone, or a combination thereof. Also, the end blocks, in one embodiment, may have a polymer composed of monomers that can be l-lactide, D-lactide, glycolide, L,D-lactide, or a combination thereof.

In another embodiment of the presently disclosed implantable medical device, the at least one inner block is a center block. Thus, for this embodiment, the block which has equal number of blocks on either side of it is the center block. As an example, when a multiblock copolymer is represented by ABBCBBA, C is the center block and A serves as both end blocks.

It is possible in one embodiment, when there is a center block, a majority of (greater than 50%) the center block monomer composition is from monomers selected from the group consisting of e-caprolactone, r-butylactone, trimethylene carbonate, caprolactone derivatives, P-Dioxanone, or a combination thereof. Alternatively, the center block monomer composition (with one or more monomers selected from this list) may be greater than 55%, 60%, 65%, 70%, 75%, 80%, 85%, 90% or 95%. Even for this center block however, other monomers may be added to the composition of the center block to achieve different properties such as faster degradation, certain level of strength. The other monomers that can be added may be selected from the group consisting of l-lactide, D-lactide, glycolide, and L, D-lactide or a combination thereof. These would be in the minority (less than 50%) and alternatively each less than 45%, 40%, 35%, 30%, 25%, 20%, 15%, 10%, or 5%.

Moreover, when there are end blocks, it is possible that for each end block, a majority of (greater than 50%) the end block monomer composition is from monomers selected from the group consisting of l-lactide, D-lactide, glycolide, and L, D-lactide. Alternatively, the end block monomer composition (with one or more monomers selected from this list) may be greater than 55%, 60%, 65%, 70%, 75%, 80%, 85%, 90% or 95%. Even for these end blocks however, other monomers may be added to the composition of the end blocks to achieve different properties such as variable degradation rate, certain level of flexibility. These other monomers that can be added may be selected from the group consisting of e-caprolactone, r-butylactone, trimethylene carbonate, caprolactone derivatives, P-Dioxanone, or a combination thereof. These would be in the minority (less than 50%) and alternatively each less than 45%, 40%, 35%, 30%, 25%, 20%, 15%, 10%, or 5%.

In another embodiment, the end blocks are hard blocks. Hard blocks are composed of hard polymers. Hard polymers as used herein refer to polymer blocks with T_{g} higher than physiological temperatures, so the polymers behave like hard plastic. Examples of hard polymers as used herein include, but are not limited to polylactide and poly(lactide-co-glycolide).

For the present multiblock copolymers the inner blocks may either be hard or soft. In one embodiment, a center block will be soft. Soft blocks are composed of soft polymers. Soft polymers as used herein refers to polymer blocks with T_{g} lower than physiological temperatures, so the polymer is soft and flexible at such temperatures. Examples of soft polymers as used herein include, but are not limited to poly(trimethylene carbonate) and poly(ε-carprolactone).

In another embodiment, the presently disclosed implantable medical device comprises a biodegradable multiblock copolymer having at least three blocks that alternate between a hard block and a soft block. The end block can be either hard or soft. The inner blocks can be hard or soft. An inner block may be a center block. Thus, where there are seven blocks, the resulting copolymer may be represented by hard-soft-hard-soft-hard-soft-hard. In one embodiment, when the blocks alternate, the end blocks are hard and the center block (when there is one) is soft.

In another embodiment of the presently disclosed implantable medical device, the multiblock copolymer is a triblock copolymer. Here, it is also true that each of the three blocks may be a homopolymer or a copolymer.

In one embodiment, the center block of the triblock copolymer is the polymer poly(trimethylenecarbonate) and the end blocks are made of poly(L-lactide). In this embodiment then, each block is a homopolymer.

For all of the various embodiments of the presently disclosed implantable medical device, the multiblock copolymers may or may not be cross-linked. Cross-links can be formed by chemical reactions that are initiated by heat, pressure, or radiation. For example, mixing of an unpolymerized or partially polymerized resin with specific chemicals called crosslinking reagents results in a chemical reaction that forms crosslinks. Cross-linking can also be induced in materials that are normally thermoplastic through exposure to a radiation source, such as electron beam exposure, gamma-radiation, or UV light. For example, electron beam processing is used to cross-link the C type of cross-linked polyethylene. Other types of cross-linked polyethylene are made by addition of peroxide during extruding (type A) or by addition of a cross-linking agent (eg. vinylsilane) and a catalyst during extruding and then performing a post-extrusion curing.

Cross-links are the characteristic property of thermosetting plastic materials. In most cases, cross-linking is irreversible, and the resulting thermosetting material will degrade or burn if heated, without melting. Especially in the case of commercially used plastics, once a substance is cross-linked, the product is very hard or impossible to recycle. In some cases, though, if the cross-link bonds are sufficiently different, chemically, from the bonds forming the polymers, the process can be reversed. Permanent wave solutions, for example, break and re-form naturally occurring cross-links (disulfide bonds) between protein chains in hair.

Chemical covalent cross-links are stable mechanically and thermally, so once formed are difficult to break. A class of polymers known as thermoplastic elastomers rely on physical cross-links in their microstructure to achieve stability. They offer a much wider range of properties than conventional cross-linked elastomers because the domains which act as cross-links are reversible, so can be reformed by heat.

Exemplary cross-linkers which may be used in accordance with the scope and teachings of the present disclosure are various diisocyanates.

In another embodiment, the end blocks may serve as one or more cross-linking points.

In another embodiment of the presently disclosed implantable medical device, the multiblock copolymer is an elastomer. An elastomer is a polymer with the property of elasticity. The term, which is derived from elastic polymer, is often used interchangeably with the term rubber, and is preferred when referring to vulcanisates. Each of the monomers which link to form the polymer is usually made of carbon, hydrogen, oxygen and/or silicon. Elastomers are amorphous polymers existing above their glass transition temperature, so that considerable segmental motion is possible. At ambient temperatures rubbers are thus relatively soft (E˜3 MPa) and deformable. Elastomers are usually thermosets (requiring vulcanization) but may also be thermoplastic (see thermoplastic elastomer). The long polymer chains cross-link during curing. The molecular structure of elastomers can be imagined as a ‘spaghetti and meatball’ structure, with the meatballs signifying cross-links. The elasticity is derived from the ability of the long chains to reconfigure themselves to distribute an applied stress. The covalent cross-linkages ensure that the elastomer will return to its original configuration when the stress is removed. As a result of this extreme flexibility, elastomers can reversibly extend from 5-700%, depending on the specific material. Without the cross-linkages or with short, uneasily reconfigured chains, the applied stress would result in a permanent deformation. Temperature effects are also present in the demonstrated elasticity of a polymer. Elastomers that have cooled to a glassy or crystalline phase will have less mobile chains, and consequentially less elasticity, than those manipulated at temperatures higher than the glass transition temperature of the polymer. It is also possible for a polymer to exhibit elasticity that is not due to covalent cross-links, but instead for thermodynamic reasons.

Further, the properties of the present multiblock copolymers are the result of the monomers which make up the copolymers. Also, the reaction conditions employed in making the instant multiblock copolymers which include, but not limited to, temperature, solvent choice, reaction time and catalyst choice, can be varied to affect the properties of the multiblock copolymers. Varing the monomer ratios allows one of ordinary skill in the art to fine tune, or modify the properties of the multiblock copolymers to control various physical properties.

In another embodiment of the present disclosure, implantable medical devices may have the instant multiblock copolymers as one or more coatings. These coatings may or may not further comprise at least one bioactive agent. However, more generally, the present implantable medical devices may contain one or more bioactive agents even when the instant multiblock copolymers are not used as a coating.

Thus, an embodiment of the presently disclosed implantable medical device further comprises at least one bioactive agent. The term “bioactive agent(s)” as used herein refers to any organic, inorganic, or living agent that is biologically active or relevant. For example, a bioactive agent can be a protein, a polypeptide, a polysaccharide (e.g. heparin), an oligosaccharide, a mono- or disaccharide, an organic compound, an organometallic compound, or an inorganic compound. It can include a biologically active molecule such as a hormone, a growth factor, a growth factor-producing virus, a growth factor inhibitor, a growth factor receptor, an anti-inflammatory agent, an antimetabolite, an integrin blocker, or a complete or partial functional insense or antisense gene. It can also include a man-made particle or material, which carries a biologically relevant or active material. An example is a nanoparticle comprising a core with a drug and a coating on the core. Such nanoparticles can be post-loaded into pores or co-deposited with metal ions.

Bioactive agents also can include drugs such as chemical or biological compounds that can have a therapeutic effect on a biological organism. Bioactive materials include those that are especially useful for long-term therapy such as hormonal treatment. Examples include drugs for contraception and hormone replacement therapy, and for the treatment of diseases such as osteoporosis, cancer, epilepsy, Parkinson's disease and pain, an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, and combinations thereof. Suitable biological materials further can include, without limitation, an anti-restenotic agent, an anti-inflammatory agent, an HMG-COA reductase inhibitor, an antimicrobial agent, an antineoplastic agent, an angiogenic agent, an anti-angiogenic agent, a thrombolytic agent, an antihypertensive agent, an anti-arrhythmic agent, a calcium channel blocker, a cholesterol-lowering agent, a psychoactive agent, an anti-depressive agent, an anti-seizure agent, a contraceptive, an analgesic, a bone growth factor, a bone remodeling factor, a neurotransmitter, a nucleic acid, an opiate antagonist and combinations thereof. Additional bioactive materials include, without limitation, paclitaxel, rampamycin, everolimus, tacrolimus, zotarolimus, sirolimus, des-aspartate angiotensin I, nitric oxide, apocynin, gamma-tocopheryl, pleiotrophin, estradiol, aspirin, statin, atorvastatin, cerivastatin, fluvastatin, lovastatin, pravastatin, rosuvastatin, simvastatin, and combinations thereof.

Bioactive agent(s) also can include precursor materials that exhibit the relevant biological activity after being metabolized, broken-down (e.g. cleaving molecular components), or otherwise processed and modified within the body. These may include such precursor materials that might otherwise be considered relatively biologically inert or otherwise not effective for a particular result related to the medical condition to be treated prior to such modification.

Combinations, blends, or other preparations of any of the foregoing examples can be made and still be considered bioactive agents within the intended meaning herein. Aspects of the present invention directed toward bioactive agents can include any or all of the foregoing examples.

Bioactive agents may elute from the present implantable medical devices. One non limiting method for altering the bioactive agent elution profile from the implantable medical devices is to mix different block copolymer components including the presently disclosed multiblock copolymers in different ratios. For example, mixtures of different polymers and/or copolymers having differing hydrophilicities and hydrophobicities can significantly affect a coating's performance. Another method for tuning a polymer/block copolymer (as used herein polymer tuning refers to a process of adjusting a polymer's composition to achieve a desired elution profile and other physical characteristics) is to alter the individual monomers that comprise a given polymer or block copolymer. Thus, polymer scientists may experiment using condensation and addition techniques to tune specific polymers. While condensation and addition techniques are useful with relative simple polymers, more complex polymer structures can be achieved using these methods. This is also true when polymers are used in biomedical applications where the multi-factorial demands on a polymer's performance are critical. The present inventors turned to block copolymers as a possible alternative to polymer coatings derived from blending a limited number of miscible polymers and copolymers and/or being limited to the few existing block copolymers made using the teachings of the prior art such as those disclosed in U.S. Pat. No. 6,855,770 which is incorporated herein by reference for all it contains regarding block copolymers. Methods were needed that permitted the use of a wider range of monomer subunits, combinations of polymers and bioactive agents and more production friendly manufacturing techniques.

In another embodiment, the present implantable medical device may be a vascular stent, stent graft, urethral stent, biliary stent, suture, ocular device, heart valve, shunt, pacemaker, bone screw, bone anchor, protective plate, or prosthetic device, or even combinations thereof. A preferred embodiment is a vascular stent. When it is placed inside a patient, it made degrade partially or fully. A vascular stent may completely be made of the instant multiblock copolymers or include them within a stent framework made of something else, such as metal. When a vascular stent has a metal stent framework, the metal may comprise a material selected from the group consisting of stainless steel, nitinol, tantalum, MP35N alloy, platinum, titanium, a biocompatible alloy, a biocompatible polymer and a combination thereof. Alternatively, the vacular stents as disclosed herein may further comprise polymers which are not the presently disclosed multiblock copolymers. They may include but are not limited to urethanes, polylactides, poly-l-lactic acids, polyglycolic acids, polycaprolactones, polyacrylates, polymethacrylates, polymethylmethacrylates, and combinations and/or compolymers thereof.

In another embodiment, the presently disclosed implantable medical devices are self-expanding. Self-expanding implantable medical devices as used herein elastically resist compression in a free state, or are radially expandable where they are expanded using an expansion device such as a balloon catheter. An embodiment of a self-expanding implantable medical device is a vascular stent which is self-expanding. Clearly, a sheath mechanism is not necessary if an expansion device is required to expand the stent. Ideally, the self-expanding stents are formed from a material which offers resistance to the pressure of a beating heart which pressure might otherwise plastically deform less resistant materials. Such materials include nitinol, nickel free self-expanding alloys or self-expanding polymers including the present multiblock copolymers.

Any diol, polyethylene glycol, dextran and other polysaccharides may be used as an initiator for polymerization for the center block. The catalyst may be a tin catalyst or other metal catalysts known such as tin octoate.

Polymerization of the presently disclosed multiblock copolymers may be a one step polymerization or a two step polymerization. In the one step polymerization, the center block is polymerized and the monomers for the end blocks are added directly into a reaction vessel without terminating the polymerization of the center block. This can be used when the center block polymer can be easily mixed with the added monomers. In the circumstance where it is difficult to mix the center block polymer with the added monomers, the polymerization of the center block may be terminated. The polymer may then be purified and dried. The center block polymer may be mixed with monomers by dissolving them in a common solvent. After mixing, solvent may be removed thoroughly and a new catalyst may be added for polymerization.

### EXAMPLES

### Synthesis of Triblock Copolymers

1. Polymerize trimethylene carbonate using tin octoate and 1,8 octanediol as catalysts at 130° C. for 24 hours.
2. Mix poly(trimethylene carbonate) (“PTMC”) with L-lactide and glycolide well.
3. Add a tin catalyst and begin polymerization at 130° C.
4. The polymer is then dissolved in dichloromethane and precipitated into methanol.

TABLE 1
Various Physical Characteristics of Triblock Copolymers:

| | | | Tensile | | | | |
|---|---|---|---|---|---|---|---|
| | | | stress | Tensile | Tensile | Tensile | Tensile |
| | | Modulus | at Yield | strain at | stress at | strain at | stress at |
| | | (Automatic | (Zero | Yield (Zero | Break | Break | Maximum |
| | Specimen | Young's) | Slope) | Slope) | (Cursor) | (Cursor) | Load |
| | label | (MPa) | (MPa) | (%) | (MPa) | (%) | (MPa) |
| Dry sample | | | | | | | |
| test dry | B11 | 2315 ± 45 | 48 ± 1 | 2.95 ± 0.06 | 40 ± 3 | 200+* | 48 ± 1 |
| | B12 | 2963 ± 108 | 64 ± 3 | 2.9 ± 0.1 | 47 ± 9 | 18 ± 22 | 64 ± 3 |
| | B16 | 1853 ± 346 | 39 ± 7 | 2.9 ± 0.1 | 30 ± 7 | 142 ± 78 | 39 ± 7 |
| | B17 | 2248 ± 195 | 52 ± 2 | 3.0 ± 0.2 | 40 ± 5 | 170 ± 88 | 52 ± 2 |
| | B18 | 1980 ± 121 | 48 ± 2 | 3.1 ± 0.1 | 47 ± 4 | 258 ± 30 | 49 ± 2 |
| Wet | | | | | | | |
| sample* | | | | | | | |
| test wet* | B11 | 1107 ± 62 | 24 ± 7 | 2.9 ± 0.3 | 28.6 ± 0.6 | 200+* | 28.7 ± 0.5 |
| | B12 | 1798 ± 258 | 28 ± 3 | 3.0 ± 0.2 | 21.8 ± 0.1 | 200+* | 28 ± 3 |
| | B16 | 1570 ± 117 | 24 ± 1 | 2.71 ± 0.04 | break out of | 23.8 ± 0.8 | 60.48% |
| | | | | | range | | |
| | B17 | 1654 ± 41 | 26 ± 2 | 2.87 ± 0.08 | break out of | 26 ± 2 | 50.53% |
| | | | | | range | | |
| | B18 | 1649 ± 58 | 27 ± 1 | 2.8 ± 0.2 | break out of | 27.3 ± 0.5 | 56.74% |
| | | | | | range | | |

*Wet samples were soaked in PBS at 37 degrees Celsius for 24 hr.
*Wet test is done in water bath at 37 degrees Celsius.
*200+: the sample elongates beyond the instrument limit which is 200%

TABLE 2
Molecular Weight Characterization of Block Copolymers

| Specimen | | | |
|---|---|---|---|
| Label | Mn (g/mol) | Mw (g/mol) | Polydispersity |
| B16-1942-22 | 174063 | 282479 | 1.62 |
| B17-1942-22 | 180679 | 313739 | 1.74 |
| B18-1942-22 | 219385 | 363009 | 1.65 |

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.”

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. An implantable medical device comprising:
a biodegradable multiblock copolymer comprising at least three blocks;
wherein said at least three blocks includes at least one inner block and two end blocks;
further wherein each of said at least one inner block comprises monomers selected from the group consisting of e-caprolactone, r-butylactone, trimethylene carbonate, caprolactone derivatives, P-Dioxanone, and combinations thereof; and
further wherein each of said end blocks comprises monomers selected from the group consisting of l-lactide, D-lactide, glycolide, and L,D-lactide, and combinations thereof.

2. The implantable medical device of claim 1, wherein one of said at least one inner block is a center block.

3. The implantable medical device of claim 1, wherein each block is a homopolymer.

4. The implantable medical device of claim 1, wherein each block is a copolymer.

5. The implantable medical device of claim 2, wherein said center block is a soft block.

6. The implantable medical device of claim 1, wherein each of said end blocks is a hard block.

7. The implantable medical device of claim 2, wherein said at least three blocks alternate between a hard block and a soft block; and further wherein said center block is a soft block; and further wherein each of said end blocks is a hard block.

8. The implantable medical device of claim 1, wherein said multiblock copolymer is a triblock copolymer.

9. The implantable medical device of claim 8, wherein the center block is poly(trimethylenecarbonate) and the end blocks are poly(L-lactide-co-glycolide).

10. The implantable medical device of claim 1, wherein said biodegradable multiblock copolymer is cross-linked.

11. The implantable medical device of claim 1, wherein said end blocks serve as one or more cross-linking points.

12. The implantable medical device of claim 1, wherein said multiblock copolymer is an elastomer.

13. The implantable medical device of claim 1, further comprising at least one bioactive agent.

14. The implantable medical device of claim 13, wherein said at least one bioactive agent is selected from the group consisting of an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, and combinations thereof.

15. The implantable medical device of claim 13, wherein said bioactive agent is zotarolimus.

16. The implantable medical device of claim 1, wherein said implantable medical device is selected from the group consisting of a vascular stent, stent graft, urethral stent, biliary stent, suture, ocular device, heart valve, shunt, pacemaker, bone screw, bone anchor, protective plate, prosthetic device, and combinations thereof.

17. The implantable medical device of claim 1, wherein said implantable medical device is a vascular stent.

18. The implant medical device of claim 1, wherein said implantable medical device is self-expanding.

19. A self-expanding vascular stent consisting essentially of:
a biodegradable triblock copolymer consisting essentially of
a poly(trimethylene carbonate) center block and two poly(L-lactide-co-glycolide) end blocks.

20. The self-expanding vascular stent of claim 19, further consisting essentially of a bioactive agent.
